(19) United States
(12) Patent Application Publication
(10) Pub. No.: US 2006/0207604 A1
(43) Pub. Date: Sep. 21, 2006

(54) Intubation device and method of use

(75) Inventors: Lindsey A. Nelson, New Richmond, OH (US); Douglas R. Mongeon, Orange, CA (US)

Correspondence Address: MIND LAW FIRM, 3140 RED HILL AVENUE, SUITE 150, COSTA MESA, CA, 92626, US

(73) Assignee: RADLyn LLC

(21) Appl. No.: 11/437,208

(22) Filed: May 19, 2006

Related U.S. Application Data
(63) Continuation-in-part of application No. 10/455,929, filed on Jun. 6, 2003.

(51) Int. Cl. A62B 9/06; A61M 16/00

(52) U.S. Cl. 128/207.14; 128/200.26; 128/207.15; 128/207.16

(57) ABSTRACT

The present invention is a method of performing endotracheal intubation and a new and novel intubation device for performing the method. In an exemplary embodiment of the invention, the intubation device comprises an endotracheal tube having a forward open end and a rearward open end and an inner bore there between. A stylet is telescopingly positioned within the inner bore and extends outwardly from the forward open end and includes a longitudinally extending soft and flexible guide portion. A distal stylet cuff may be inflated to provide a smooth transition from the guide portion to the endotracheal tube, and a proximal unidirectional end cap may engage a proximal connector of the endotracheal tube and cooperate with the cuff to seat the stylet within the endotracheal tube for intubation as a unitary whole.

### RELATED APPLICATIONS

This is a continuation-in-part application of a prior filed and currently pending application having Ser. No. 10/455,929 and a filing date of Jun. 6, 2003.

### BACKGROUND OF THE INVENTION

1. Incorporation by Reference

Applicant hereby incorporates herein by reference any and all U.S. patents and U.S. patent applications cited or referred to in this application.

2. Field of the Invention

Aspects of this invention relate to an intubation tube and more particularly, to a new and improved intubation device having a stylet for use in placing an endotracheal tube into the trachea of a patient.

3. Description of Related Art

Intubation devices, such as oral-endotracheal tubes, are utilized in a wide variety of medical situations to provide an unobstructed conduit to a patient's trachea in which oxygen, medications and therapies can be passed. In most situations in which medical attention is needed, medical personnel will first determine the ability of the patient to maintain their airway. Often these emergency evaluations are performed by individuals having inadequate training and knowledge in airway management. The inability to secure an airway and provide a route of ventilation in someone whose airway is threatened, compromised, or obstructed can quickly result in irreversible brain damage and death.

Placement of an intubation device, such as an endotracheal tube, into the tracheobronchial tree of a patient is accessed via the nasal or oral opening. In a few select situations nasal intubation is indicated, however, the vast majority of airways are secured via the oraltracheal route. Placement of an endotracheal tube, however, is often found to be a difficult procedure, even by well-trained, experienced personnel. This difficulty can be attributed to the anatomical variations or the encountered situation in which the airway needs to be secured. Further, during the process of placing an endotracheal tube into the trachea of a patient, it is not uncommon that only minimal visualization of the laryngeal anatomy can be obtained thereby increasing the chances that esophageal intubation can occur. Thus, the ability to “blindly” place an endotracheal tube properly into the trachea of a patient is directly proportional to the medical personnel's experience and the availability of additional intubation devices.

Endotracheal tubes are generally formed of soft, pliable plastic materials that increase in plasticity in the warm humidified air encountered when passing through the hypopharynx, thereby increasing the difficulty of placing an endotracheal tube through the glottic opening even under the most optimal visualization. Making the endotracheal tube out of a stiffer material, however, is not an acceptable alternative because it would cause excessive trauma to the nasal or throat tissue as the laryngeal and surrounding mucosa is extremely sensitive to manipulation. This can result in significant swelling and decreased visualization, which prevents or hinders oral tracheal intubation thereby placing the patient's life in great jeopardy. To overcome this problem, intubation devices often have a removable stylet made out of a somewhat malleable material that is telescopically placed within the endotracheal tube and has a sufficient amount of stiffness in order to maintain the tube's desired contour and to aid in tracheal intubation.

The stylet, which has gained the most acceptance for oral intubation, is a “pre-bend” stylet made of a rigid, malleable material such as rubber or plastic coated metal. Typically, during the intubation process, the medical personnel performing the process inserts the stylet into the endotracheal tube and folds one end of the stylet around the outboard end of the tube. The tube and the stylet are then molded to approximately conform to what is believed to be the route of passage to the laryngeal inlet. With the help of a laryngoscope, the medical personnel inserts the stylet and the endotracheal tube into the patient's mouth and hypopharynx until it reaches the patient's trachea. If needed, after visualizing the hypopharynx, the tube and stylet can be removed and the contour adjusted if necessary. Unfortunately, it is difficult for medical personnel, even after extensive training, to predetermine the proper contour that the endotracheal tube should be molded to for placement into the patient's trachea. Accordingly, it is not uncommon to fail in the first attempt to intubate the patient. In such an event, medical personnel must then remove the tube and the stylet from the patient, adjust their contour, re-ventilate the patient, and reinsert the now adjusted tube and stylet into the patient. Such removal and reinsertion of the endotracheal tube and stylet results in valuable time lost and may also, result in damage to the patient's soft airway tissue thereby significantly increasing the likelihood of loss of visualization of the laryngeal anatomy and the time for oxygen delivery. Further, sterility of the endotracheal tube may be sacrificed when the user grasps the stylet to rebend the stylet into the proper contour.

In order to overcome some of the problems encountered with such intubation devices, mechanical guides have been developed to assist intubation of endotracheal tubes. It has been found that such mechanical guides are typically difficult to manipulate with one hand and do not allow for delicate control or provide the proper sensitivity necessary to intubate a patient quickly with a minimum amount of trauma to sensitive tissue. Accordingly, such mechanical guides have not met with widespread commercial success or recognition in the medical field and the malleable or “prebend” stylet is still the predominate oral intubation aid used. Further, such guides are also typically formed from materials that are sufficiently rigid to maintain a desired contour. Accordingly, such guides have been found to cause trauma to sensitive tissue.

It is therefore desirable to have an intubation device that can be quickly and easily inserted to form an unobstructed conduit to a patient's trachea in which oxygen, medications and therapies can be passed, that reduces the likelihood of trauma to the sensitive nasal or throat tissue of the laryngeal and surrounding mucosa, that reduces the chances of compromising the sterility of the device, and which can be relatively inexpensive to manufacture.

### SUMMARY OF THE INVENTION

Aspects of the present invention include a method of performing endotracheal intubation and a new and novel intubation device for performing the method. In a preferred embodiment t of the invention, the intubation device for use in medical intubation comprises an endotracheal tube having a forward open end and a rearward open end and an inner bore there between. The endotracheal tube is provided with a fitting having a neck portion for inserting longitudinally within the rearward open end and an adaptor for connecting to a respirator or other ventilating apparatus. A stylet is slidably and telescopingly positioned within the inner bore and includes a proximal end that extends outwardly from the rearward open end and adaptor and a distal end having a longitudinally extending more soft and semi-flexible guide portion. In a preferred embodiment, the stylet is adapted to slide or move outwardly from the forward open end of the endotracheal tube such that during insertion of the endotracheal tube the guide portion moves towards the larynx until it is positioned within the trachea. Thereafter, the endotracheal tube telescopingly slides or moves forwardly over the stylet and guide portion which operates to direct the endotracheal tube into proper position within the trachea. The stylet can then be removed by withdrawing the stylet rearwardly out through the rearward open end of the endotracheal tube and fitting. The endotracheal tube can then be attached to a respirator or other ventilating apparatus using the adaptor.

In another preferred embodiment of the invention, the intubation stylet includes a sheath-covered wire assembly having an inflation line assembly installed at its proximal (non-patient) end and a flexible guide tip assembly installed at the opposite, distal (patient) end. A unidirectional end cap is positioned on the wire substantially at the proximal end of the stylet. An inflation cuff is installed substantially at the distal end of the stylet so as to generally overlap the interface between the sheath-covered wire and the flexible guide tip preferably, the proximal end cap and the inflation cuff cooperate during use to secure the stylet within an endotracheal tube or other intubation device so that the two devices may then be intubated as a unitary whole.

In another preferred embodiment of the invention, an endotracheal tube is provided with an inflatable bladder or balloon that when inflated prevents ventilation gas flowing through the endotracheal tube from escaping outwardly from the trachea.

In another preferred embodiment of the invention, the stylet comprises a semi-flexible metal rod or wire.

In another preferred embodiment of the invention, the semi-flexible metal rod is formed from copper.

In another preferred embodiment of the invention, the metal rod is coated by a soft and pliable plastic, such as a polyethylene material.

In another preferred embodiment of the invention, the stylet is tapered to minimize the lip formed between the forward open end of the endotracheal tube and the stylet.

In another preferred embodiment of the invention, the forward open end of the endotracheal tube includes a cuff to minimize the lip formed between the forward end of the endotracheal tube and the stylet.

In another preferred embodiment of the invention, the endotracheal tube includes an inner sleeve adapted for receiving the stylet to permit the endotracheal tube and stylet to be bent into a desired contour with greater unity.

In another preferred embodiment of the invention, the intubation device of the present application further comprises means for inducing curvature to the endotracheal tube and stylet.

In another preferred embodiment of the invention, the stylet includes a longitudinally extending hollow core having a proximal open end and a distal open end adapted to connect to a standard oxygen supply.

In another preferred embodiment of the invention, the stylet includes a light effective for aiding in the intubation of the patient.

In another preferred embodiment of the invention, the stylet sheath is formed with a secondary lumen in communication with the stylet cuff.

In another preferred embodiment of the invention, the stylet cuff is formed with a proximal annular portion and a distal annular portion, the proximal annular portion being sealably installed on the stylet wire assembly and the distal annular portion being sealably installed on the flexible guide tip assembly.

In another preferred embodiment of the invention, the flexible guide tip assembly comprises a tip sheath having a hollow bore terminating in a distal closed end and a tip wire inserted within the hollow bore.

In another preferred embodiment of the invention, the stylet wire assembly includes a stylet wire installed within a primary lumen of the stylet sheath, the stylet wire being configured so as to not extend to the distal end of the stylet sheath, and the tip sheath is formed with a proximally-facing shoulder configured to engage the distal end of the stylet sheath during assembly of the flexible guide tip assembly on the stylet wire assembly.

In another preferred embodiment of the invention, the inflation line assembly is sealably installed in fluid communication with the proximal opening of the secondary lumen of the stylet sheath.

In another preferred embodiment of the invention, the unidirectional end cap is formed with at least one living hinge oriented so as to allow distal movement of the unidirectional end cap along the stylet wire assembly and so as to discourage proximal movement of the unidirectional end cap along the stylet wire assembly.

In another preferred embodiment of the invention, the stylet wire assembly includes a stylet wire installed within the primary lumen of the stylet sheath, the stylet wire being configured so as to not extend to the distal end of the stylet sheath, and the distal tip of the stylet sheath being closed and rounded, whereby a distal atraumatic flexible guide tip is formed.

In another preferred embodiment of the invention, the overall length of the stylet cuff ranges from approximately one to three inches.

In an preferred embodiment of the invention, the method of performing endotracheal intubation of a patient comprises the steps of first inserting an endotracheal tube and a stylet of an intubation device through the mouth of a patient and down the throat towards the larynx. The stylet is then slid or moved outwardly from the endotracheal tube such that the soft and semi-flexible guide portion of the stylet enters the trachea. The stylet then operates to direct the endotracheal tube into proper position within the trachea. The stylet is then removed out through the rearward open end of the endotracheal tube and the endotracheal tube is then attached to a respirator or other ventilating apparatus.

In another preferred embodiment of the invention the curvature of the stylet is adjusted to a desired contour for insertion into the trachea of the patient.

In another preferred embodiment of the invention, the stylet includes a chemiluminescent light which is bent to activate the chemiluminesent light.

In another preferred embodiment of the invention, an inflatable bladder or balloon is inflated to prevent ventilation gas flowing through the endotracheal tube from escaping outwardly from the trachea.

In another preferred embodiment of the invention, the method of intubating a patient comprises the steps of inserting an intubation stylet within an intubation device, inflating a stylet cuff installed substantially on the distal end of the intubation stylet so as to seat the intubation stylet within the intubation device and form a tapered transition from a distal flexible guide tip of the intubation stylet to the distal tip of the intubation device, and passing the intubation stylet and intubation device into the trachea of the patient.

In another preferred embodiment of the invention, a unidirectional end cap mounted substantially on the proximal end of the intubation stylet is engaged with a connector installed substantially on the proximal end of the intubation device.

In another preferred embodiment of the invention, the stylet cuff is deflated, the intubation stylet is advanced through the intubation device such that the flexible guide tip and the stylet cuff are temporarily passed out of the distal end of the intubation device until the unidirectional end cap engages the connector, and the stylet wire assembly is retracted relative to the unidirectional end cap until the stylet cuff is properly positioned adjacent the intubation device.

In another preferred embodiment of the invention, a mark on the intubation stylet is aligned with the distal tip of the intubation device so as to position the stylet cuff adjacent the intubation device.

In another preferred embodiment of the invention, an intubation device cuff is inflated so as to seat and substantially seal the intubation device within the trachea, the stylet cuff is deflated, and the intubation stylet is withdrawn proximally by disengaging the unidirectional end cap from the connector.

Other embodiments and advantages of the invention will be apparent from the following description, the accompanying drawings and the appended claims.

### BRIED DESCRIPTION OF THE DRAWINGS

To provide a more complete understanding of the present invention and further features and advantages thereof, reference is now made to the following description taken in conjunction with the accompanying drawings, in which:

FIG. 1 is a partial view showing the oral passageway of a patient for the exchange of gasses between the lungs and the outside atmosphere and showing a preferred embodiment of the intubation device of the present invention partially inserted;

FIG. 2 is a side view of the intubation device of FIG. 1;

FIG. 3 is a side view of the intubation device of FIG. 1 showing the stylet in its unsheathed position within the endotracheal tube;

FIG. 4 is a rearward sectional view of the intubation device of FIG. 1 taken along line 4-4 of FIG. 3 with the stylet positioned in its sheathed position within the endotracheal tube and showing the stylet and the soft and pliable plastic sleeve;

FIG. 5 is a partial view showing oral intubation of a patient utilizing the intubation device of the present invention with the stylet in its extended position within the trachea;

FIG. 6 is a partial view showing oral intubation of a patient utilizing the intubation device of the present invention with the endotracheal tube within the trachea and the inflatable bladder inflated to prevent ventilation gas flowing through the endotracheal tube from escaping outwardly from the trachea;

FIG. 7 is a rearward sectional view of another preferred embodiment of the invention showing the inner wall of the endotracheal tube having an inner sleeve adapted for receiving the stylet;

FIG. 8 is a partial view of another preferred embodiment of the invention showing the endotracheal tube and the stylet in its unsheathed position and having a taper for minimizing the lip formed between the forward open end of the endotracheal tube and the stylet;

FIG. 9 is a forward end view of the stylet of FIG. 8 taken along line 9-9;

FIG. 10 is a partial view of another preferred embodiment of the invention showing the forward open end of the endotracheal tube having a cuff for minimizing the lip formed between the forward open end of the endotracheal tube and the stylet;

FIG. 11 is a rearward sectional view of the stylet of FIG. 10 taken along line 11-11 showing the stylet having a longitudinally extending hollow core;

FIG. 12 is a partial side view of the guide portion of the stylet showing a chemiluminescent light;

FIG. 13 is a perspective view of another preferred embodiment of the intubation device of the present invention showing the intubation stylet having a distal cuff and a proximal unidirectional end cap;

FIG. 14 is an enlarged partial sectional view of the intubation device of FIG. 13 taken along line 14-14 showing the cuff installed on the stylet so as to be in communication with the secondary lumen of the stylet for inflation and deflation of the cuff;

FIG. 15 is an enlarged sectional view of the intubation device of FIG. 13 taken along line 15-15 showing the sheath with secondary lumen installed on the stylet wire;

FIG. 16 is an enlarged partial perspective view of the intubation device of FIG. 13 showing the unidirectional end cap and inflation line assembly;

FIG. 17 is an enlarged partial perspective view of the intubation device of FIG. 13, partially in section, showing the interior space formed between the flexible tip assembly and the cuff;

FIG. 18 is a reduced scale side view of the intubation device of FIG. 13 showing the stylet partially inserted within the endotracheal tube;

FIG. 19 is a reduced scale side view of the intubation device of FIG. 13 showing the stylet fully inserted within the endotracheal tube and the cuff inflated to form a smooth transition between the flexible tip of the stylet and the wall of the endotracheal tube;

FIG. 20 is a reduced scale side view of the intubation device of FIG. 13 showing the stylet fully inserted within the endotracheal tube, the cuff inflated, and the unidirectional end cap advanced to engage the connector on the endotracheal tube;

FIG. 21 is a reduced scale side view of the intubation device of FIG. 13 showing the stylet fully inserted within the endotracheal tube and engaging the endotracheal tube through the inflated cuff and the end cap, with the resulting intubation device being formed to a selected curvature for insertion into the patient's trachea;

FIG. 22 is a partial view of the oral passageway of a patient showing the intubation device of FIG. 13 configured as shown in FIG. 21 now partially inserted within the oral passageway, the distal tip of the stylet just passing through the vocal cords;

FIG. 23 is a partial view of the oral passageway of a patient showing the intubation device of FIG. 13 configured as shown in FIG. 21 now fully inserted within the oral passageway, the distal ends of the stylet and endotracheal tube having passed through the vocal cords and into the trachea, with the endotracheal tube cuff there inflated to secure the endotracheal tube in position and to prevent ventilation gas flowing through the endotracheal tube from escaping outwardly from the trachea; and

FIG. 24 is a partial view of the oral passageway of a patient showing the intubation device of FIG. 13 with the endotracheal tube secured with the trachea and the stylet cuff deflated and the stylet partially withdrawn proximally out of the endotracheal tube.

### DETAILED DESCRIPTION OF THE INVENTION

The present invention relates to a method of performing endotracheal intubation and a new and novel intubation device for performing the method. In describing the preferred embodiments of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose.

For purposes of the description of the present invention, the terms “distal”, “forward” and “forwardly” are intended to refer to the direction towards the patient receiving the intubation device, whereas the terms “proximal”, “rear” and “rearwardly” are intended to refer to the direction away from the patient receiving the intubation device.

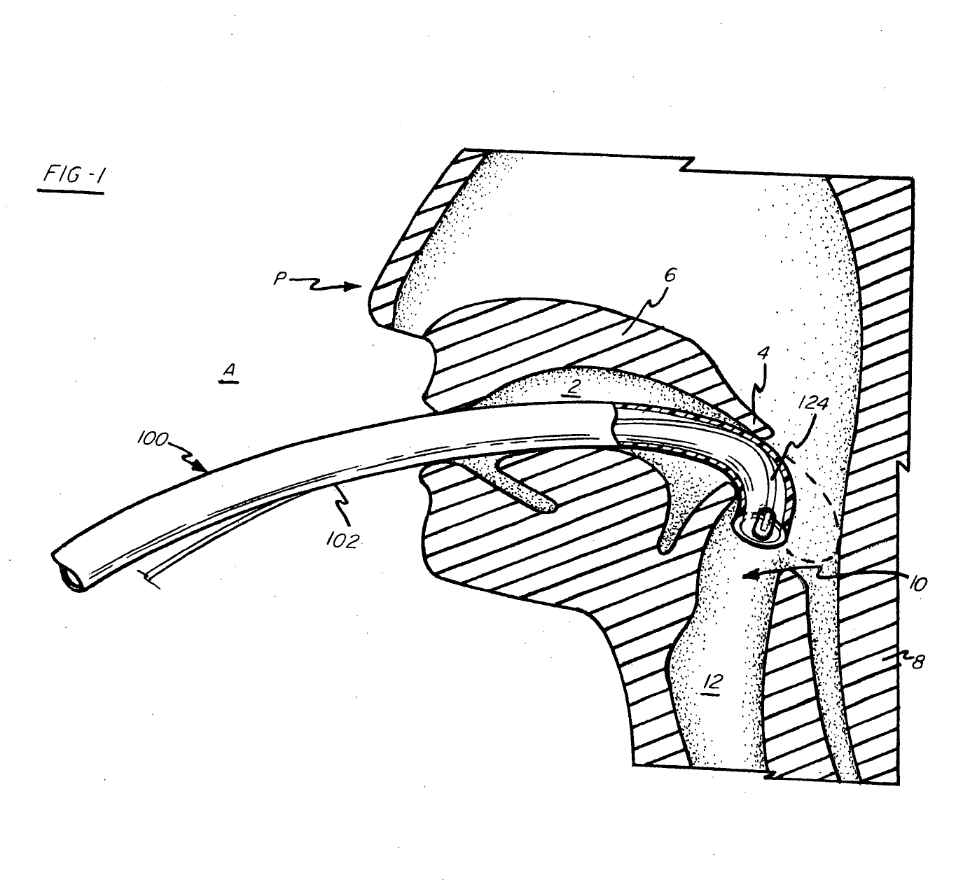

Referring to FIG. 1, a partial cross-section of a patient P is shown illustrating the mouth 2, the epiglottis 4, the nasopharynx 6, the esophagus 8 that operate to transfer food to the stomach (not shown), the larynx 10, and the trachea 12 that operate to provide a passageway for the exchange of gasses between the lungs (not shown), the alveoli (not shown), and the outside atmosphere A.

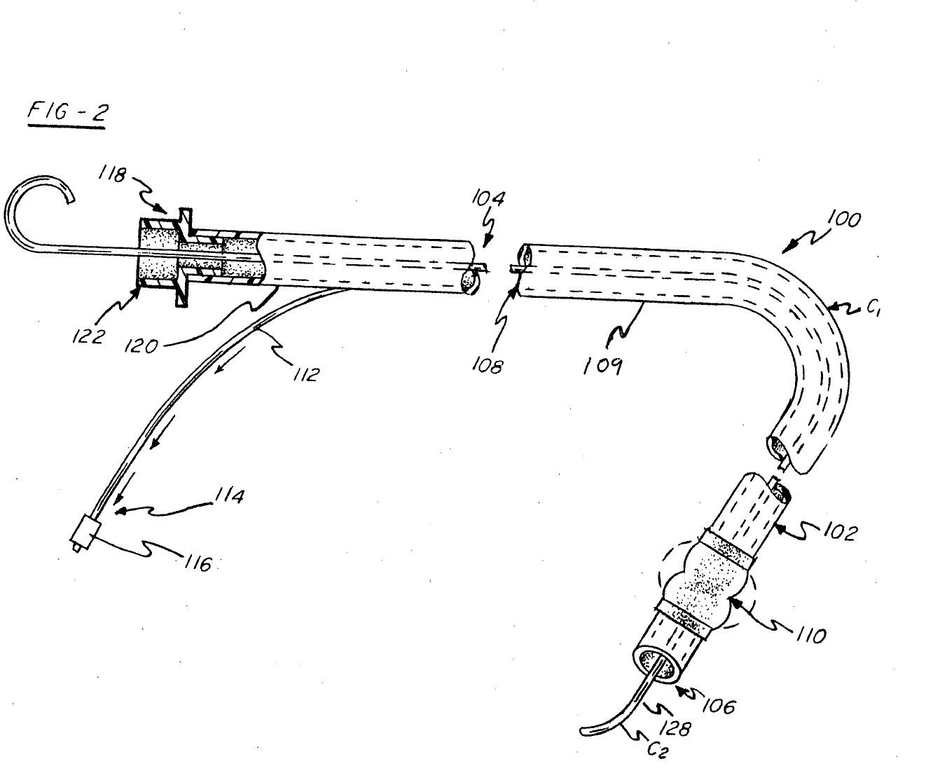

Referring to FIG. 2, a preferred embodiment of the intubation device 100 of the present application is shown comprising an elongated endotracheal tube 102 having a rearward open end 104 and a forward open end 106 having a longitudinal bore 108 there between. The endotracheal tube 102 is formed of a pliable semi-rigid, soft plastic material such as, but not limited to, a polyethylene, a polypropylene, or like material. The lower forward end of the endotracheal tube 102 includes one or more inflatable bladders or balloons 110 which is attached to an air device through an air line 112 such that when inflated the bladder 110 operates to prevent ventilation gas flowing through the endotracheal tube 102 from escaping outwardly from the trachea 12 of the patient P. A portion of the air line 112 is preferably positioned within the wall 109 of the endotracheal tube 102 and provides flow communication between the bladder 110 and an external air source (not shown). The rearward end 114 of the air line 112 is provided with a flow valve 116 to permit a syringe (not shown) or other inflation device to be placed in flow communication with the air line 112 for injecting a predetermined amount of air into the bladder 110.

The rearward open end 104 of the endotracheal tube 102 is provided with a fitting 118 having a neck portion 120 for inserting longitudinally within the rearward open end 104 and an adaptor 122 for connecting to a respirator or other ventilating apparatus or oxygen supply, anesthesia supply, or some other medical gas supply.

Referring to FIGS. 2, 3 and 4, telescopically positioned within the endotracheal tube 102 is an elongated stylet 124 which is formed from a semi-flexible material that can be bent relatively easily into a desirable contour, such as AWG 6-14 copper wire of a circular cross section or some other suitable material that can be bent into a desired contour and rigid enough to support and help maintain the endotracheal tube 102 in a desired contour. The stylet 124 includes a soft and pliable sleeve 126 formed from a plastic, such as, but not limited to, a polyethylene, polypropylene, polyvinylchloride, or the like, or a rubber composition. In a preferred embodiment, the sleeve 126 covers the entire length of the stylet 124 and extends longitudinally outwardly from the forward end of the stylet 124 to form a soft, more flexible and more malleable guide portion 128. The rearward end of the stylet 124 extends longitudinally outwardly through the endotracheal tube 102 and fitting 118 such that medical personnel can easily grasp the end of the stylet 124 during the intubation procedure. Preferably, the rearward end of the stylet 124 can be curved or looped, as shown, or have a larger cross section, or can include a collar or grip to provide a better gripping surface for medical personnel. Further, the forward ends of the endotracheal tube 102 and the stylet 124 are preferably rounded or tapered to minimize trauma to the tissue of the patient's airway during insertion.

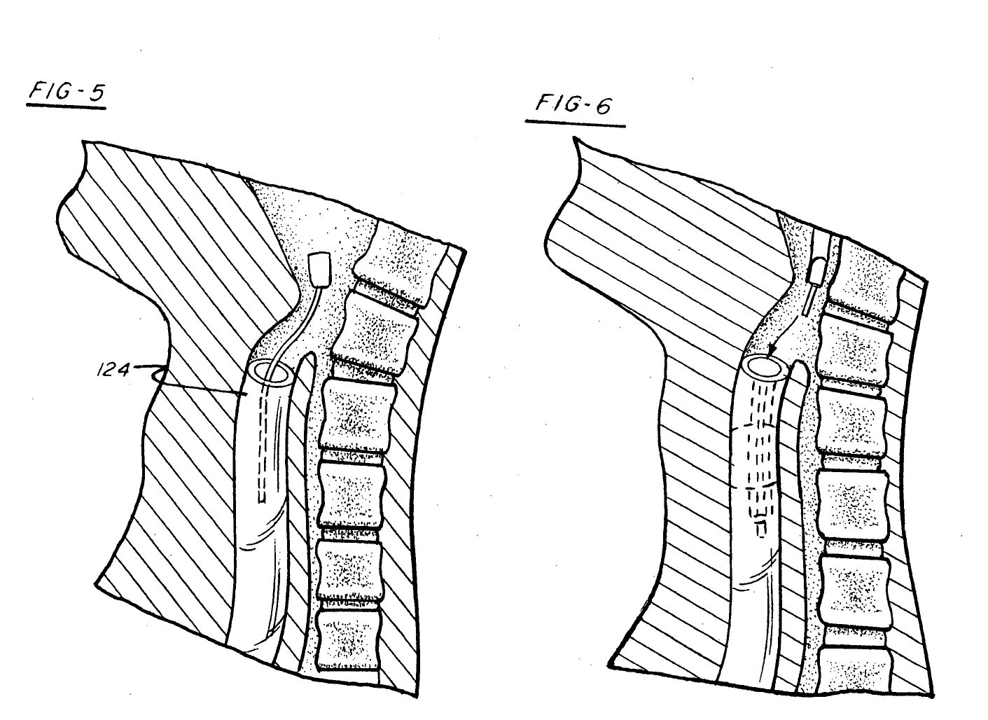

Referring to FIGS. 1, 5, and 6, in operation of a preferred embodiment of the present invention, the intubation device 100 is first bent into the desired contour (step 1) that the medical personnel believes approximates what is believed conforms to the patient's air passage way to the trachea 12. The intubation device 100 is then inserted into the patient's mouth 2 (step 2) such that the endotracheal tube 102 together with the stylet 124 are moved behind the epiglottis 4 past the esophagus 8 (FIG. 1) and towards the larynx 10. In order to aide in insertion of the endotracheal tube 102 and to avoid trauma to sensitive throat tissue, after the forward end of the intubation device 100 approaches the larynx 10, the medical personnel moves the stylet 124 forwardly such that the guide portion 128 moves longitudinally outwardly from the endotracheal tube 102 and forwardly towards and through the larynx 10 and into the trachea 12 (step 3) (FIG. 5). It should now be apparent to those skilled in the art that by reducing the risk of tissue trauma often encountered with first insertion of an endotracheal tube 102 into a patient's trachea 12, tissue swelling around the trachea 12 is minimized allowing medical personnel an unobstructed view of the larynx 10. After the guide portion 128 of the stylet 124 has been properly inserted into the trachea 12, the endotracheal tube 102 is telescopically slid or moved forwardly along the stylet 124 and guide portion 128 through the larynx 10 and into position within the trachea 12 (step 4) (FIG. 6).

Once the endotracheal tube 102 is in position within the trachea 12, air is injected through the flow valve 116, such as by a syringe, to inflate the bladder 110 (step 5). The inflated bladder 110 then operates to provide a seal to eliminate any gas that is being supplied by the endotracheal tube 102 from passing back out through the larynx 10. Medical personnel can then remove the stylet 124 (step 6) by retracting the guide portion 128 rearwardly out through the larynx 10 and out through the rearward open end 104 and fitting 118. The endotracheal tube 102 can then be connected using the adaptor 122 to a respirator or other ventilating apparatus or oxygen supply, an anesthesia supply, or some other medical gas supply. After use, the endotracheal tube 102 can be removed by first releasing air from the bladder 110 by opening the flow valve 116 and slowly withdrawing the endotracheal tube 102. The intubation device 100 is disposable and can be discarded after use.

It should be understood that the endotracheal tube 102 and stylet 124 can have a predetermined curvature to aide in the easy and quick insertion of the intubation device 100. For example, as shown in FIG. 2, the endotracheal tube 102 and stylet 124 can have a first curvature, such as shown at C1, and a second curvature, such as shown at C2. It should now be understood by those skilled in the art, however, that the endotracheal tube 102 and stylet 124 can be formed into a variety of curvatures or molded by hand to a desirable curvature. It should also now be understood by those skilled in the art that during the insertion process, if necessary, the stylet 124 can be removed and its contour adjusted and reinserted into the endotracheal tube 102 thereby adjusting its contour.

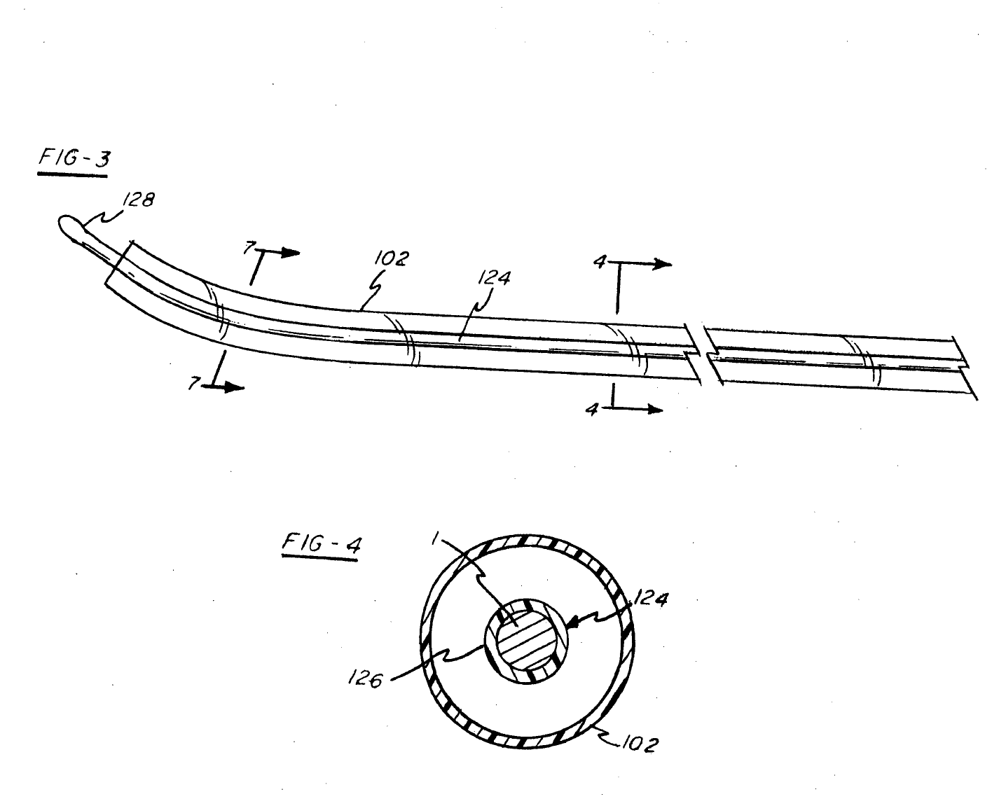

Referring now to FIGS. 3 and 7, a rearward end sectional view of another preferred embodiment of the intubation device 100 of the present application is shown whereby the endotracheal tube 102 includes an inner sleeve 130 (not shown in FIG. 3) that runs longitudinally along the inner wall 132 of the endotracheal tube 102 and is adapted for receiving stylet 124. In operation, the endotracheal tube 102 and stylet 124 operate as previously described, but it should now be apparent to those skilled in the art that placement of the stylet 124 within the inner sleeve 130 will permit the endotracheal tube 102 and stylet 124 to be bent into a desired contour with greater unity. In this way, the endotracheal tube 102 can be more easily bent and maintained in the desired contour. It should be understood that the inner sleeve 130 may be a singular unitary sleeve or may be formed from a plurality of cuffs and the like.

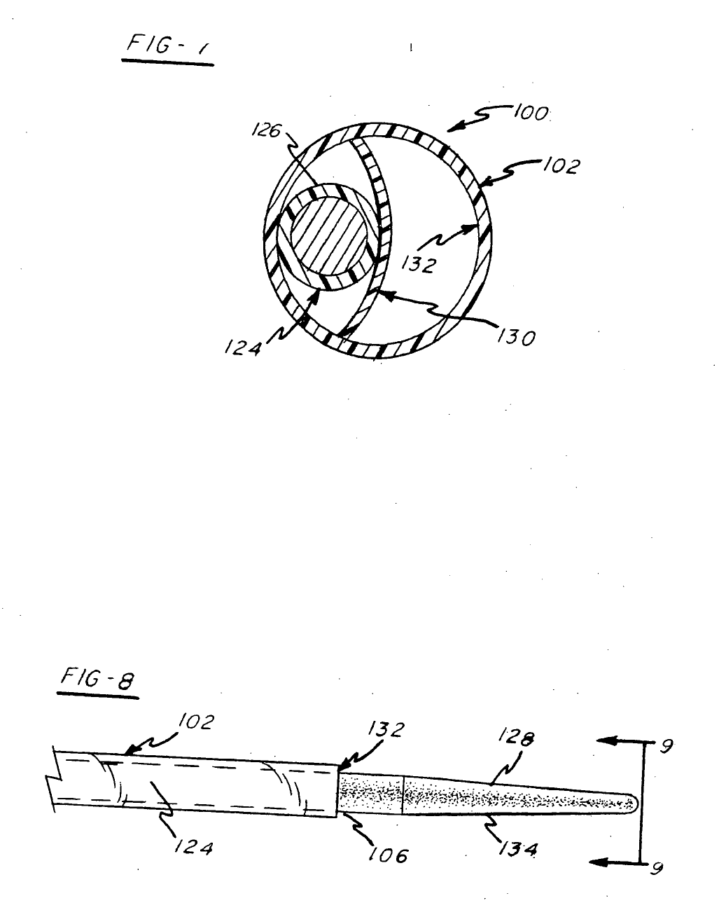

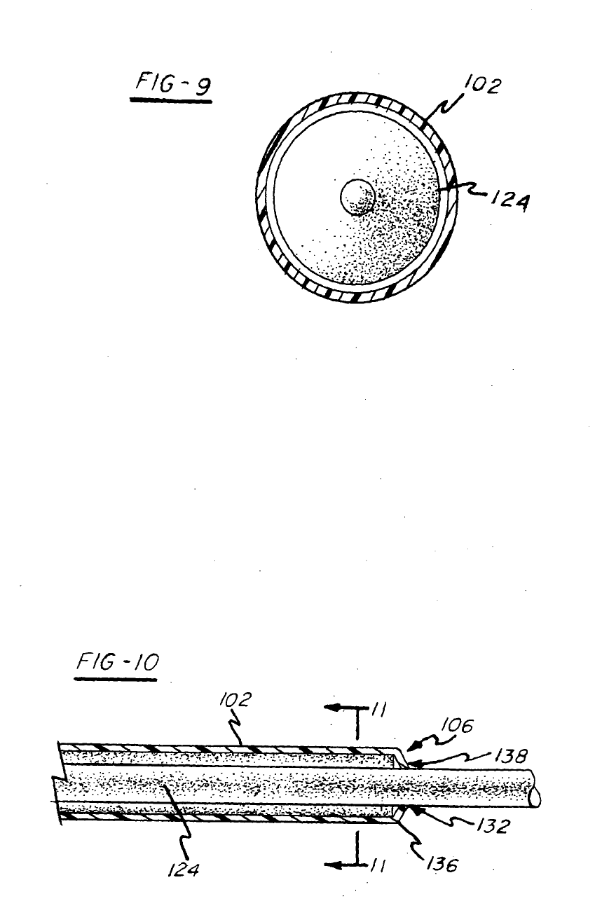

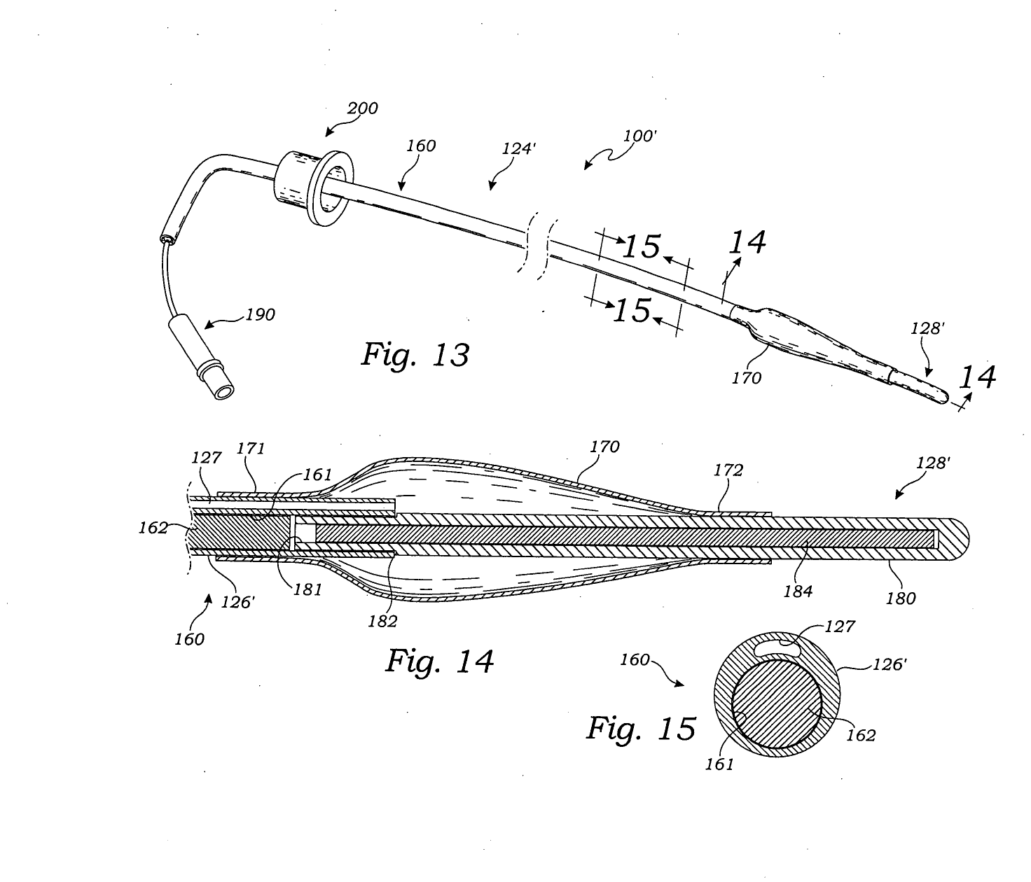

Referring to FIGS. 8 and 9, the stylet 124 is shown in its unsheathed position. It should be understood by those skilled in the art that during insertion of the intubation device 100, sharp edges may result in trauma to sensitive tissue. Accordingly, it is desirable to minimize the lip 132 formed between the endotracheal tube 102 and the stylet 124 when the stylet is in its unsheathed position. In a preferred embodiment of the invention shown, the diameter of the forward end of the stylet 124 decreases in a forwardly direction towards the guide portion 128 forming a taper portion 134. As shown, when the stylet 124 is extended in its unsheathed position, the taper operates to minimizing the lip 132 formed between the forward open end 106 of the endotracheal tube 102 and the stylet 124. It should also be understood that the taper portion 134 may also be formed by providing the guide portion 128 with an expandable portion such that when expanded, such as by air being directed downward through the stylet 124, tapers the forward end of the stylet 124, as explained more fully below in connection with other embodiments of the present invention, such as shown in FIGS. 13-24.

Referring to FIG. 10, another preferred embodiment of the invention is shown whereby the forward open end 106 of the endotracheal tube 102 includes and thin plastic or rubber membrane 136 forming a collar 138 around the stylet 124. As shown, the collar 138 operates to minimize the lip 132 formed between the endotracheal tube 102 and the stylet 124.

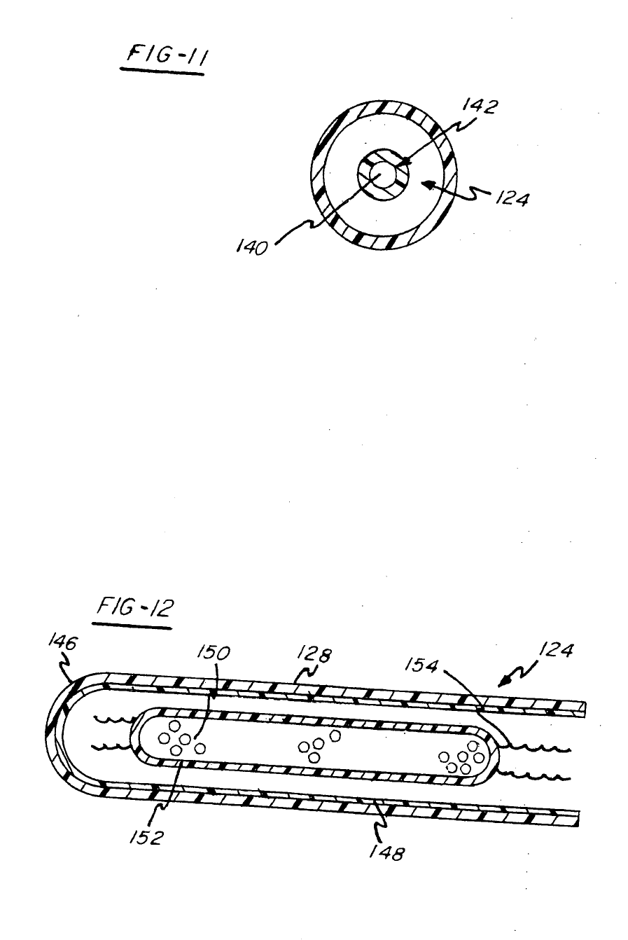

Referring to FIGS. 1 and 11, a rearward end view of another preferred embodiment of the present invention is shown whereby the stylet 124 has a longitudinally extending hollow core 140 that runs the entire length of the stylet 124 having a rearward first open end 142 and a forward second open end (not shown). The first end 142 of the stylet 124 is adapted to connect to a standard oxygen supply (not shown) for ventilation. During operation, once the stylet 124 is inserted into the trachea 12, oxygen can be immediately supplied to the patient P. The endotracheal tube 102 can then be telescopically moved over the stylet 124 and into the trachea 12. The stylet 124 can then be removed. It should be apparent to those skilled in the art that the ability to inject oxygen through the stylet 124 can provide critically needed oxygen to the patient in the event insertion of the endotracheal tube 102 meets with an unexpected obstruction or resistance.

It should also now be apparent to those skilled in the art that the intubation device of the present application can be easily adapted to accommodate other various mechanisms known in the art to aide in intubation of a patient. One such mechanism is shown and described in U.S. Pat. No. 5,259,377 and is incorporated herein by reference whereby the intubation device includes a mechanism comprising a flexible member positioned within the endotracheal tube that is operated by a handle means for inducing curvature to the endotracheal tube and stylet. Another such mechanism is shown and described in U.S. Pat. No. 6,539,942 and is incorporated herein by reference whereby a control wire and handgrip is provided to curve the endotracheal tube and stylet. It should be understood, however, that mechanisms for aiding in inducing curvature of the endotracheal tube and stylet are not limited to the above examples. In another exemplary embodiment of the method of the present application, once the endotracheal tube 102 has been partially inserted into the air passageway of the patient P, if medical personnel find that the endotracheal tube 102 and stylet 124 do not have the proper contour, they can be adjusted into the desired contour by the adjustment mechanism. It should also be understood that the endotracheal tube 102 can remain inserted into the air passageway of the patient P and the stylet 124 removed and its contour adjusted into a desired configuration and reinserted back into the endotracheal tube 102 to adjust its contour into the desired configuration.

Referring to FIGS. 1 and 12, another preferred embodiment of the invention is shown comprising a light formed within the guide portion 128. In an exemplary embodiment, the light is enclosed within the guide portion 128 of the stylet 124 and comprises a vial 146 preferably formed from a pliable plastic, having a glass liner 148 containing a chemiluminescent light producing liquid reactant 150, such as bis (2, 4, 5-trichloro-6-carbopentoryphenyl) oxalate and a fluorescer in fibutyl phthalate. Positioned within the vial 146 is a sealed glass ampule 152 having an oxidizer 154, such as an 85 percent solution of hydrogen peroxide in dimethyl phthalate and a catalytic quantity of catalyst, such as sodium salicylate. In operation, prior to insertion of the intubation device 100, medical personnel activates the chemiluminescent light by bending or pressing the guide portion encompassing the vial 146 such that the glass ampule 152 breaks to allow the oxidizer 154 to mix with the reactant 150 thereby producing chemiluminescent light. In this way direct illumination of the larynx 10 and trachea 12 will be obtained.

While the intubation device and method of the present invention has been shown and described as being inserted into the patient's air pathway by way of the mouth, it should now be understood that the intubation device may also be inserted through the nose of the patient.

Turning now to FIGS. 13-24, there is shown another preferred embodiment of the intubation device 100′ of the present invention. It will be appreciated that the intubation device, as that term is used throughout, may or may not include an endotracheal tube or other such device and, as such, that the invention is not so limited. Referring to FIGS. 13-24, there is shown another preferred embodiment illustrating stylet 124′ and method for achieving an intubation of an endotracheal tube 102′ more safely and effectively. Moreover, it will be appreciated by those skilled in the art that other intubation and airway devices beyond endotracheal tubes, both now known or later developed, may be placed within a patient airway or trachea employing the device and method of the present invention, such that the following illustrated use of an endotracheal tube is merely exemplary.

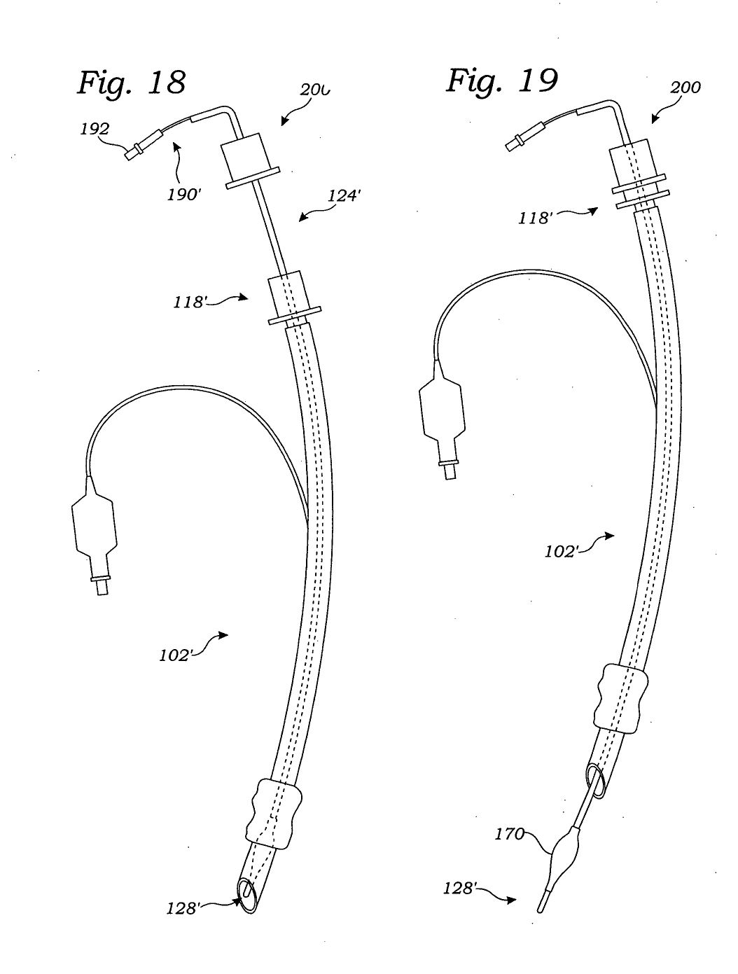

Referring first to FIG. 13, stylet 124′ is shown as comprising a sheath-covered wire assembly 160 having an inflation line assembly 190 installed at its proximal (non-patient) end and a more flexible guide tip 128′ installed at the opposite, distal (patient) end. A unidirectional end cap 200 is positioned on the wire assembly 160 substantially at the proximal end of the stylet 124′. An inflation cuff 170 is installed substantially at the distal end of the stylet 124′ so as to generally overlap the interface between the wire assembly 160 and the more flexible guide tip 128′. As explained more fully below, the proximal end cap 200 and the distal inflation cuff 170 cooperate during use to secure the stylet 124′ within an endotracheal tube 102′ (FIG. 18) or the like so that the two devices may be intubated as a unitary whole.

Turning to FIG. 14, there is shown an enlarged sectional view of the distal end of the stylet 124′. The flexible guide tip 128′ generally comprises a radiused outer plastic sheath 180 within which a small wire 184 is inserted. In the exemplary embodiment, the wire is 302 stainless steel having an overall length of about 2¾″ and a nominal diameter of 1/32″, though it will be appreciated that numerous sizes and materials are possible without departing from the scope of the invention. The wire 184 is inserted within a hollow bore 181 of the outer plastic sheath 180 substantially until it bottoms at the distal, radiused end. At the open, proximal end of the sheath 180 there is provided a proximally-facing, stepped shoulder 182 for the purpose of installing the guide tip assembly 128′ onto the stylet wire 162, more about which is said below. Preferably, when the tip wire 184 is inserted fully its proximal end is within the bore 181 and not protruding from the sheath 180, which also has advantages in assembly and use. Once more, in a further alternative embodiment, the flexible tip 128 may be achieved by extending the sheath 126′ some distance beyond the internal metal stylet wire 162 and radiusing or forming the distal tip of the sheath 126′ so as to be closed and atraumatic, much like the distal tip of the separate guide tip 128′. Those skilled in the art will appreciate that numerous other configurations of the flexible guide tip are possible without departing from the spirit and scope of the invention. It will be further appreciated that any such guide tips may be practiced in the present invention with or without the stylet cuff 170, the unidirectional end cap 200 or the stylet wire assembly 160 as shown and described. Accordingly, it is to be understood that the subject invention is not limited to the various elongated flexible tips and their use as presented herein. Referring again to FIG. 14 showing another preferred embodiment wherein the guide tip 128′ is separately installed on the stylet wire assembly 160, the stylet sheath 126′ is configured to extend distally beyond the distal end of the stylet wire 162, such that the length of the extending portion of the stylet sheath 126′ substantially corresponds to the stepped portion of the outer plastic sheath 180 up to the proximally-facing step shoulder 182. As such, surface-to-surface contact is achieved between the inside surface of the stylet sheath 126′ and the outside surface of the outer plastic sheath 180 in the region of the proximally-facing step shoulder 182. The installation of the guide tip 128′ onto the wire assembly 160 is preferably achieved through a solvent bond, though an interference or press fit, ultrasonic weld, or other such mechanical assembly means now known or later developed in the art may be employed.

With continued reference to FIGS. 14 and 15, the wire assembly 160 is shown as including the stylet sheath 126′ having a lengthwise secondary lumen 127. The sheath 126′ is preferably formed from a polypropylene, polyethylene or other such biocompatible material as through an extrusion process. As best shown in FIG. 15, the cross-section of the primary lumen 161 into which the stylet wire 162 is inserted is substantially round, while the profile of the secondary lumen 127 is shown as being somewhat crescent shaped, though it will be appreciated by those skilled in the art that all such cross-sections are merely exemplary and that the invention is not so limited. In the exemplary embodiment wherein the secondary lumen 127 is formed with the illustrated somewhat crescent cross-section, it is noted that the portion of the wall of the stylet sheath 126′ in which the secondary lumen 127 is formed is relatively thicker so as to accommodate this extrusion. Or, put another way, the primary lumen 161 is off-center from or not concentric with the cross-section of the sheath 126′. Again, those skilled in the art will appreciate that numerous-other shapes, wall thicknesses, configurations and the like for the stylet sheath and its one or more lumens are possible without departing from the spirit and scope of the invention. For example, it is also possible to employ the primary lumen 161 as the air passage as well, so that no secondary lumen is needed, either by extruding a channel (not shown) in one wall of the primary lumen 161 or simply sizing the inside diameter of the sheath to have sufficient clearance over the outside diameter of the stylet wire 162 so that air can travel therealong. Or, again, the stylet wire 162 itself could be formed with a hollow core through which the air can pass, once more eliminating the need for a secondary lumen in the stylet sheath 126′. In any such case, the distal end of the sheath 126′ in the vicinity of the stylet cuff 170 could then be formed with a channel, notch, hole or other opening (not shown) or simply not be bonded to the flexible guide tip 128′ about its complete circumference, whereby air can pass between the primary lumen 161 or hollow core of the stylet wire 162 and the interior space of the cuff 170 for inflation and deflation thereof. Once more, those skilled in the art will appreciate that the invention is not limited to the specific embodiments described herein. Regarding the wire assembly 160 more generally, the stylet wire 162 is preferably formed from steel or copper rod having a nominal 1/16″ diameter with the tubular sheath 126′ made of polyethylene and configured to slide over the stylet wire 162 to form the wire assembly 160.

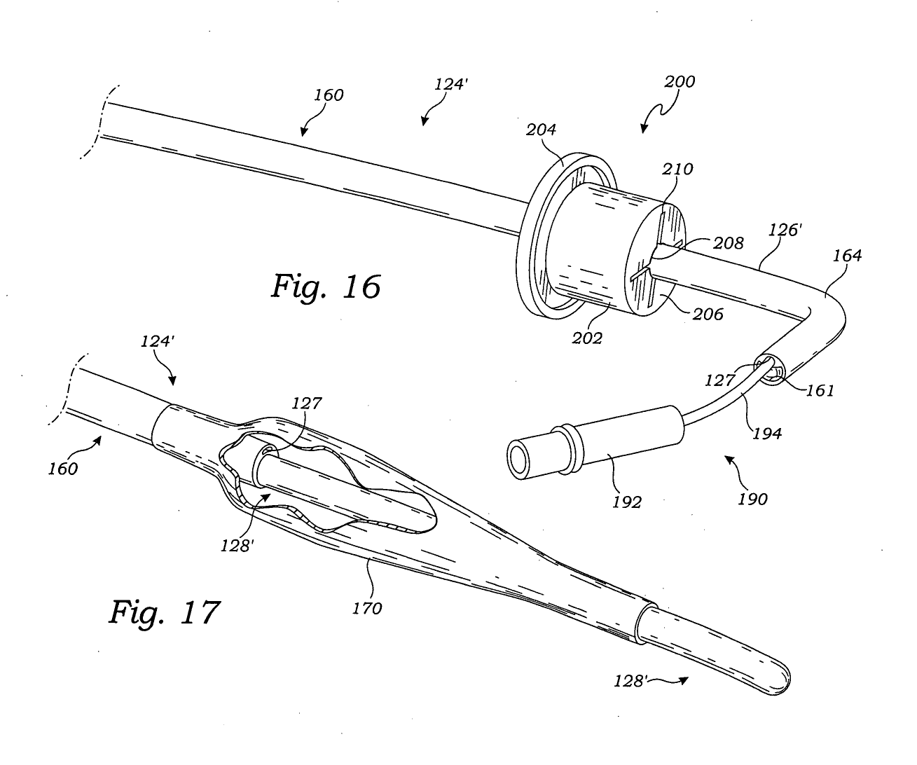

Turning to FIG. 16, an enlarged partial perspective view of the proximal end of the stylet 124′ of FIG. 13, the inflation line assembly 190 is shown as including a one-way check-valve 192 and a small inflation line 194. The inflation line 194 is installed in the proximal end of the secondary lumen 127 (FIG. 14) so as to be in fluid communication therewith. This installation is accomplished by solvent bonding or by any other assembly means now known or later developed. By configuring the inflation line assembly 190 with a one-way valve 192, as is known in the art, it will be appreciated that air can be introduced through the valve 192 into the secondary lumen 127 without any escaping. As such, with reference to FIG. 17, any air so introduced into the secondary lumen 127 through the valve 192, as through a syringe, will then pass along the entire length of the lumen 127 to its proximal end and there pass into the interior space bounded by the cuff 170 so as to inflate the cuff 170 during use. Alternately, by means of this same fluid path, with the connection of a syringe or the like to the valve 192 so as to actuate it, as is known in the art, the air within the cuff 170 and the secondary lumen 127 can then be withdrawn, as when the cuff 170 is to be deflated and the stylet 124′ withdrawn from the airway, more about which is said below. Referring again to FIG. 16, the proximal opening of the primary lumen 161 of the stylet sheath 126′ is preferably potted or otherwise sealed after the stylet wire 162 is inserted therein so as to prevent the stylet wire 162 from moving relative to the sheath 126′ and any air from escaping through the primary lumen 161 that has passed into the cuff 170 through the secondary lumen 127 as described above. In the alternative embodiments wherein the air passageway to the cuff 170 is formed from the primary lumen 161 itself or a hollow bore of the stylet wire 162, it will be appreciated that other means for installing the inflation in assembly 190 and sealing about the primary lumen 161 of the stylet sheath 126′ may be employed without departing from the spirit and scope of the invention.

With continued reference to FIG. 16, the unidirectional end cap 200 is positioned on the wire assembly 160 substantially at the proximal end, preferably prior to installation of the inflation line assembly 190 or formation of any bend in the proximal end of the wire assembly 160. The end cap 200 is configured with a tapered annular body 202 sized and configured to meet ASTM/ISO 5356 standards for 15 mm connectors used in medical breathing circuits, equipment and devices. The distal end of the cap 200 is formed with a radially-outwardly extending flange 204 to facilitate grasping the cap 200 for engagement and disengagement with other connectors. The proximal end of the end cap 200 is defined by a planar wall 206 forming the bottom surface of the inside, or female portion, of the connector and having a hole 208 for the passage therethrough of the wire assembly 160. The hole 208 is preferably sized to substantially have a net fit with the outside surface of the stylet wire assembly 160, or the sheath 126′, specifically. Furthermore, the proximal planar wall 206 of the end cap 200 is formed with a plurality of radial slots 210 each intersecting the hole 208 so as to effectively define the wall 206 as a plurality of living hinges each flexing axially substantially about the proximal perimeter of the annular body 202. While four slots 210 roughly ninety degrees (90°) apart, and thus four living hinge sections that are roughly quadrants, are shown, it will be appreciated that virtually any number of slots 210 and resulting hinge sections may be formed within the planar wall 206 without departing from the spirit and scope of the invention. In more detail, the through-hole 208 may be formed with an annular, distally projecting tubular body (not shown) substantially concentric therewith and having the same slots 210 passing therethrough, whereby these axially-extending annular wall portions cooperate with the hinge sections formed in the end wall 206 to selectively flex in and out of contact with the wire assembly 160 during use, more about which is explained below. Or, in an alternative embodiment, the wall of the hole 208 itself is simply tapered from a smaller proximal diameter to a larger distal diameter. In any such configuration, it will be appreciated by those skilled in the art that as the stylet wire assembly 160 is passed through the hole 208 proximally, or the end cap 200 is advanced along the wire assembly 160 distally, the hinge sections of the end wall 206 will flex distally, effectively opening up the hole 208 and allowing the wire assembly 160 to slide therethrough. Then, if one were to attempt to retract the end cap 200 proximally along the wire assembly 160, the end wall 206 would be forced back into a substantially planar configuration as the proximal perimeter of the hole 208 effectively engages the outer surface of the sheath 126′, thereby reducing the diameter of the hole 208 and preventing travel of the end cap 200 along the wire assembly 160 in the proximal direction. This one-way movement of the end cap 200 along the wire assembly 160 resulting from the construction of the end wall 206 with through-hole 208 and slots 210 provides several functional advantages during use, as explained more below. Those skilled in the art will appreciate that numerous other one-way, or unidirectional, mechanical arrangements involving living hinges and the like, now known or later developed, may be employed in the end cap 200 of the present invention and that the exemplary embodiment of the end cap 200 shown and described is merely illustrative. Once the end cap 200 is so configured on the wire assembly 160, a bend 164 may be formed in the proximal end of the wire assembly 160 between the end cap 200 and the proximal end of the device, or the inflation line assembly 190, to aid in manipulation of the stylet 124′ during use. While a roughly ninety degree (90°) bend is shown in the alternative embodiment of FIGS. 13-24, it will be appreciated that a variety of angles and configurations of the proximal end of the stylet wire assembly 160 are possible without departing from the spirit and scope of the invention.

In the final assembly step best shown in FIG. 14, the inflation balloon or cuff 170 is installed near the distal end of the stylet 124′ so as to effectively span the wire assembly 160 and the flexible guide tip 128′. Thus, the inflation path mentioned above whereby, in the exemplary embodiment, air injected into secondary lumen 127 through the valve assembly 190 exits the secondary lumen 127 and inflates the cuff 170. Similarly, again, the cuff 170 can later be deflated through the same fluid path. The cuff 170 itself is preferably a polyethylene or other such biocompatible, elastic material that is formed having a generally “bullet-shaped” or tapered profile having an overall length of approximately 2¼″ with proximal and distal annular portions 171, 172 having an inside diameter substantially equivalent to the outside diameter of the respective sheaths 126′, 180 so as to be solvent bonded or otherwise sealed in those regions, thereby sealing off an air space within the cuff 170 that, again, is in communication only with the secondary lumen 127 and, thus, the fluid path leading back to the inflation line assembly 190. In the alternative embodiment wherein the flexible tip is defined by the sheath 126′ extending beyond the distal end of the stylet wire 162, holes (not shown) may be formed in the tip portion of the sheath 126′ so as to allow fluid communication between the sheath's interior lumen 161 and the cuff 170 installed longitudinally about the holes, or such that the proximal and distal annular portions 171, 172 span the region of the sheath wherein the cross-holes are formed. Those skilled in the art will once again appreciate that a variety of other configurations of the flexible tip portion 128′, and thus the fluid flow path in the region within the cuff 170, are possible without departing from the spirit or scope of the invention and, thus, that the subject invention should not be limited by the particular embodiments shown and described herein. The flexible guide tip 128′, whether a coated wire, an extension of the stylet sheath, or some other such arrangement, is preferably formed at its distal end with a rounded nose 129 and/or a lengthwise taper to aid in atraumatically passing the distal end of the guide tip 128′ through the vocal cords, or larynx, and into the laryngeal inlet. Similarly, the cuff 170 is preferably also tapered from front to back, or distal to proximal end, and is configured with its net fit annular portions 171, 172 to bond directly and smoothly to the outside surface of the guide tip 128′, or the outer plastic sheath 180, so as to form a relatively smooth and continuous surface for intubation. With the installation of the cuff 170 on the wire assembly 160 and guide tip 128′, the stylet 124′ is effectively completed and ready for use, though the device 100′ may first be subjected to inspection, packaging, sterilization or other such steps that are beyond the scope of the present invention.

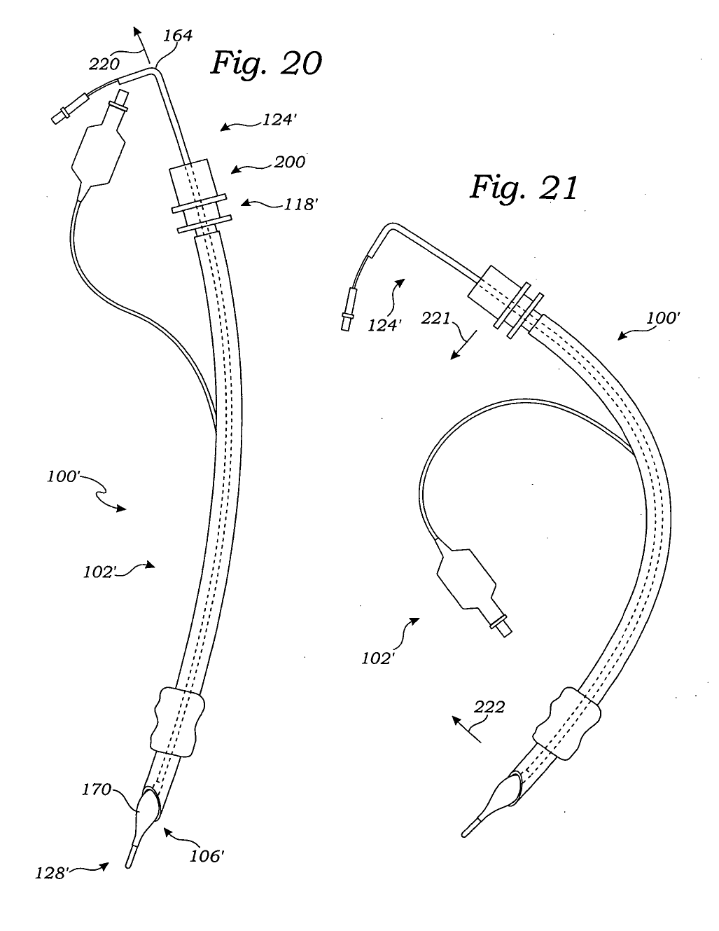

Referring now to FIGS. 18-24, the basic principles of operation of the alternative embodiment intubation device 100′ of the present invention are illustrated. To prepare the stylet 124′ for use, it is removed from its package (not shown) being careful to not pull on the inflation line assembly 190′. A syringe (not shown) is inserted into the valve 192 to withdraw all air from the device and create a vacuum in the inflatable cuff 170, pulling it tight against the outer wall of the stylet so as to minimize the device profile. With respect to the endotracheal tube 102′ through which the stylet 124′ will be passed, the inner lumen may be lubricated or rinsed with sterile water or saline to further facilitate movement of the stylet 124′ within the tube 102′. The stylet 124′ is then passed into the endotracheal tube 102′, guide tip 128′ end first, as shown in FIG. 18. In one preferred method, the stylet 124′ is advanced distally through the endotracheal tube 102′ until the unidirectional female end cap 200 engages the mating male 15 mm connector 118′ of the tube 102′, as shown in FIG. 19. Then, as shown in FIG. 20, while holding the endotracheal tube 102′ or the mating connectors 200, 118′, the stylet 124′ may be gripped on the wire assembly 160 in the vicinity of the ninety degree 90° bend 164, again, being careful not to pull on the inflation line assembly 190, and retracted proximally in the direction of arrow 220 through the unidirectional end cap 200 until a black hash mark (not shown) on the stylet 124′ in the vicinity of the cuff 170 is a ligned with the distal tip 106′ of the endotracheal tube 102′. Once more, due to the construction and function of the unidirectional end cap 200, as explained above, the stylet 124′ cannot now be advanced distally against the end cap 200 and back out of the endotracheal tube 102′. With the stylet 124′ in this position, a syringe (not shown) is again connected to the valve 192′ of the inflation line assembly 190 and the cuff 170 is now inflated as explained previously so that its diameter is roughly equal to or slightly greater than the outside diameter of the endotracheal tube 102′ at its tip 106′. Not only does this create a smooth, continuous tapered leading surface for passing through the larynx, or vocal cords, 10′ and into the trachea 12′, as described more fully below, but the inflated cuff 170 now prevents the stylet 124′ from being further retracted. Because, again, the unidirectional end cap 200 prevents further advancement of the stylet 124′, now the end cap 200 and the inflated cuff 170 cooperate to effectively prevent any further movement of the stylet 124′ relative to the endotracheal tube 102′, distally or proximally. Therefore, the stylet 124′ and endotracheal tube 102′ now form a unitary intubation device with an atraumatic leading tip consisting of the flexible guide tip 128′ and the tapered inflated cuff 170 transitioning to the endotracheal tube 102′. In an alternative exemplary method by which the combination device 100′ of FIG. 20 is configured, it may also be possible, for example, after advancing the stylet 124′ through the endotracheal tube 102′ as shown in FIG. 18, to first inflate the cuff 170 to the desired diameter, next pull back on the entire stylet 124′ device, end cap 200 included, until the cuff 170 seats in the distal end 106′ of the endotracheal tube 102′, and then advance the end cap 200 distally along the stylet wire assembly 160 until it engages the connector 118′ of the endotracheal tube 102′ by holding the end of the stylet wire assembly 160 in the vicinity of the ninety degree 90° bend 164 with one hand and the end cap 200 with the other. Or, a method that is somewhat a combination of these two exemplary approaches may be employed, wherein the cuff 170 is inflated prior to the stylet wire assembly 160 being retracted so that the location of the flexible guide tip 128′ of the stylet 124′ relative to the endotracheal tube 102′ is fixed not by lining up a mark on the cuff 170 and then inflating it, but simply by lodging or seating the pre-inflated cuff within the distal opening 106′ of the tube 102′. Those skilled in the art will appreciate that each of these approaches, as well as numerous others, for arriving at the configuration of the device 100′ shown in FIG. 20 is merely exemplary and not limiting of the invention.

Now with reference to FIG. 21, as previously disclosed in connection with the embodiment of FIGS. 1-13, the stylet 124′, and thus the endotracheal tube 102′, once configured together as in FIG. 20, can next be formed to the desired curvature for intubation, or placement within the patient airway. Grasping the combination device 100′ in the vicinity of its proximal and distal ends and bending the device 100′ generally in the direction of arrows 221, 222, the device can be conformed to a selected radius, with the stylet 124′, and the stylet wire 162 (FIG. 14), specifically, bending to and holding the endotracheal tube 102′ in such position, so that the device 100′ is now ready for patient intubation.

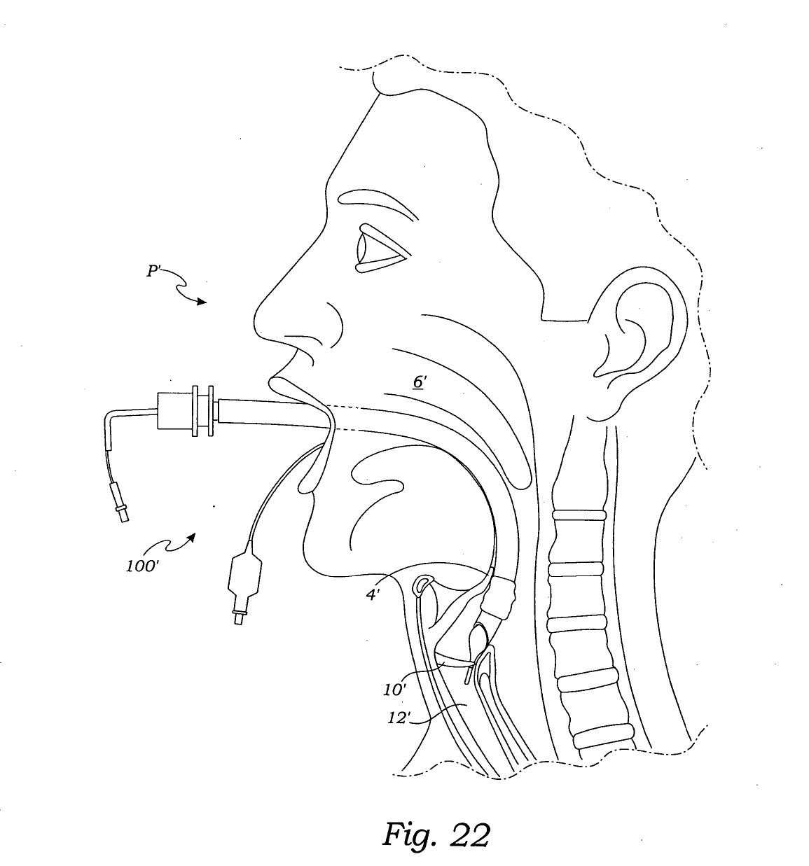

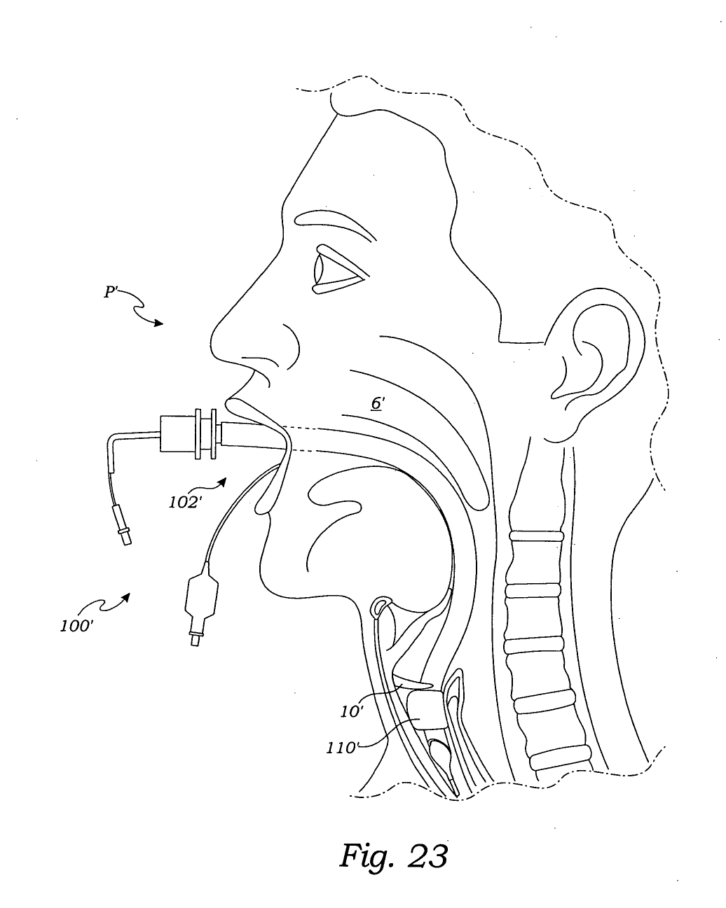

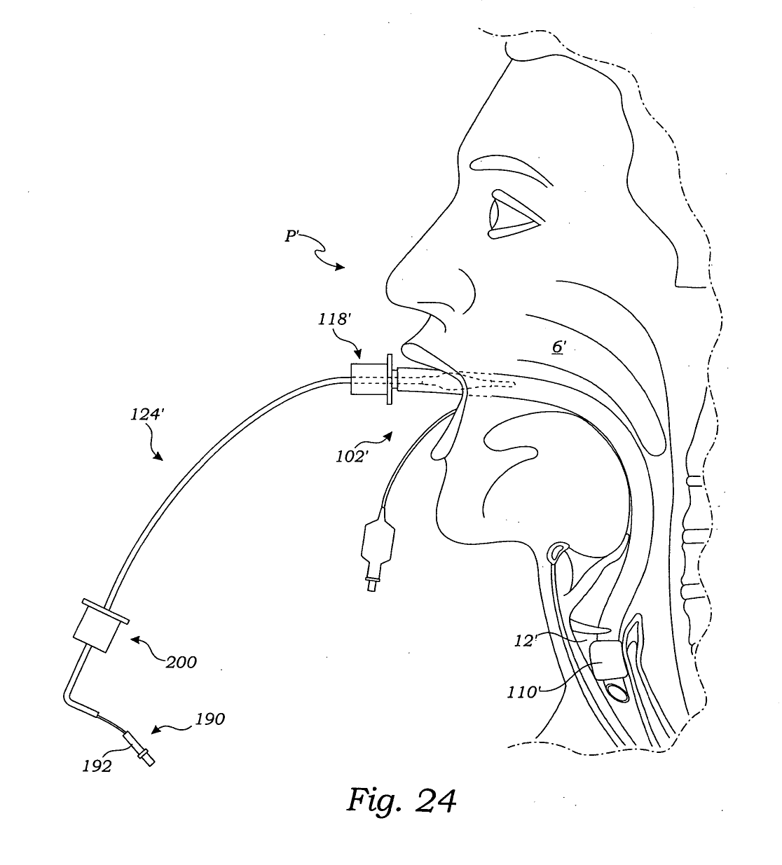

Turning, then, to FIGS. 22-24, using a laryngoscope or other such device (not shown) to expose the glottic opening, as is known in the art, the device 100′ is passed into the airway of a patient P′ through the vocal cords 10′ and into the trachea 12′. Again, a chemiluminescent light, as shown and described in connection with FIG. 12, or other such light formed in, on or about the distal end of the flexible guide tip 128′ can aid in this process in terms of visualizing the anatomy. Once more, the continuous, tapered intubation surface, resulting from the flexible guide tip 128′ and the inflated, tapered stylet cuff 170 that transitions to the outside surface of the distal end 106′ of the endotracheal tube 102′, facilitates atraumatic parting of the vocal cords 10′ and entry of the device 100′ into the trachea 12′. As distinct from the exemplary embodiment shown in FIGS. 1-12, with the alternative embodiment of the device 100′ of FIGS. 13-24, preferably the stylet 124′ is temporarily secured axially within the endotracheal tube 102′ through the cooperation of the unidirectional end cap 200 and the inflated stylet cuff 170, as described above, such that the two form effectively an integral device 100′ that is then intubated. However, those skilled in the art will appreciate that in certain circumstances it will be desirable to use the device of FIG. 20 according to the principles of operation described in conjunction with the device of FIG. 1, namely, the distal advancement of the stylet relative to the endotracheal tube so as to locate and part the larynx and then serve as a sort of guide wire to guide the endotracheal tube into the trachea, in which case the unidirectional end cap 200 would not be advanced along the stylet wire assembly 160 or engaged with the connector 118′ of the endotracheal tube 102′. With either approach, or any other such approach allowing for successful and safe intubation, once the endotracheal tube 102′ is in the correct position within the trachea 12′, as shown in FIG. 23, the tube's own cuff 110′ is inflated to establish the airway and enable positive pressure ventilation of the patient. Finally, as shown in FIG. 24, with the endotracheal tube 102′ properly positioned within the trachea 12′ of the patient P′, a syringe (not shown) or the like is again connected to the valve 192 of the inflation line assembly 190 so as to withdraw the air from the cuff 170. The unidirectional end cap 200 is then disconnected from the endotracheal tube connector 118′, and the stylet 124′ is simply withdrawn proximally through the interior lumen of the endotracheal tube 102′ and then discarded.

Once again, while the above-described method of use in various embodiments relates to the intubation of an endotracheal tube using a stylet as in the present invention, it will be appreciated by those skilled in the art that numerous other airway management devices now known or later developed may be intubated using the apparatus and method of the present invention, such that the standard endotracheal tube shown and described is merely for illustration of the principles of the invention.

It should now be apparent to those skilled in the art that the intubation device of the present application can be quickly and easily inserted to form an unobstructed conduit to a patient's trachea in which oxygen, medications and therapies can be passed. It should also now be apparent to those skilled in the art that the intubation device eliminates or reduces the problems typically associated with inducers having conventional metal stylets or the like because of the soft, flexible and malleable guide portion that reduces the likelihood of trauma to the sensitive throat tissue. It should also now be apparent to those skilled in the art that the intubation device of the present invention minimizes the lip formed between the endotracheal tube and the stylet thereby reducing the likelihood of trauma to sensitive tissue. In addition, the intubation device of the present application reduces the chances of compromising the sterility of the device, and as a disposable device can be relatively inexpensive to manufacture. It should also now be apparent to those skilled in the art that the intubation device is relatively easy to manipulate with one hand and allows for delicate control and sensitivity necessary to intubate a patient quickly with a minimum amount of trauma to sensitive tissue.

Although the foregoing invention has been described in some detail for purposes of clarity of understanding, it will be apparent that certain changes and modifications may be practiced within the scope of the appended claims. Furthermore, it should be noted that there are alternative ways of implementing both the method and article for implementing the method of the present invention. Accordingly, the present embodiments and examples are to be considered as illustrative and not restrictive, and the invention is not to be limited to the details given herein, but may be modified within the scope and equivalents of the appended claims.

What is claimed is:

1. An intubation stylet apparatus for use in medical intubation of a patient comprising:
a stylet wire assembly, including a stylet sheath having a primary lumen and a secondary lumen; and
a stylet cuff installed on the stylet wire assembly so as to be in fluid communication with the secondary lumen.

2. The apparatus of claim 1 wherein:
the stylet wire assembly has a proximal end and a distal end and the secondary lumen communicates therebetween;
a flexible guide tip is installed on the stylet wire assembly substantially at the distal end such that the distal opening of the secondary lumen is unobstructed; and
the stylet cuff is formed with a proximal annular portion and a distal annular portion, the proximal annular portion being sealably installed on the stylet wire assembly and the distal annular portion being sealably installed on the flexible guide tip, whereby fluid communication between the stylet cuff and the secondary lumen is achieved through the distal opening.

3. The apparatus of claim 2 wherein the flexible guide tip comprises:
a sheath having a hollow bore terminating in a distal closed end; and
a wire inserted within the hollow bore.

4. The apparatus of claim 3 wherein:
the stylet wire assembly further includes a stylet wire installed within the primary lumen, the stylet wire being configured so as to not extend to the distal end of the sheath; and
the sheath is formed with a proximally-facing shoulder configured to engage the distal end of the sheath during assembly of the flexible guide tip on the stylet wire assembly.

5. The apparatus of claim 2 further comprising an inflation line assembly sealably installed in fluid communication with the proximal opening of the secondary lumen, whereby fluids may be passed into and out of the cuff by means of the inflation line assembly.

6. The apparatus of claim 1 wherein:
the stylet wire assembly has a proximal end and a distal end;
the stylet cuff is installed on the stylet wire assembly substantially at the distal end; and
a unidirectional end cap is installed on the stylet wire assembly substantially at the proximal end.

7. The apparatus of claim 6 wherein the unidirectional end cap is formed with at least one living hinge oriented so as to allow distal movement of the unidirectional end cap along the stylet wire assembly and so as to discourage proximal movement of the unidirectional end cap along the stylet wire assembly.

8. The apparatus of claim 1 wherein:
the stylet wire assembly further includes a stylet wire installed within the primary lumen, the stylet wire being configured so as to not extend to the distal end of the stylet sheath; and
the distal tip of the stylet sheath is closed and rounded, whereby a distal atraumatic flexible guide tip is formed.

9. The apparatus of claim 1 wherein the stylet cuff is formed with a proximal annular portion and a distal annular portion, the stylet cuff being substantially tapered between the annular portions such that the diameter of the stylet cuff generally increases from the distal annular portion to the proximal annular portion.

10. The apparatus of claim 9 wherein the overall length of the stylet cuff ranges from approximately one to three inches.

11. An intubation stylet apparatus for use in medical intubation of a patient comprising:
a stylet wire assembly having a proximal end and a distal end;
a stylet cuff installed on the stylet wire assembly substantially at the distal end; and
a means for inflating and deflating the stylet cuff.

12. The apparatus of claim 11 wherein:
the stylet wire assembly includes a stylet sheath having a primary lumen; and
the inflation means comprises a secondary lumen formed within the stylet sheath offset from the primary lumen so as to communicate between the proximal end and the distal end of the stylet wire assembly.

13. The apparatus of claim 12 wherein:
the distal opening of the secondary lumen is in fluid communication with the stylet cuff; and
an inflation line assembly is sealably installed in fluid communication with the proximal opening of the secondary lumen, whereby fluids may be passed into and out of the cuff by means of the inflation line assembly and the secondary lumen.

14. The apparatus of claim 12 wherein:
a flexible guide tip is installed on the stylet wire assembly substantially at the distal end so that the distal opening of the secondary lumen is unobstructed; and
the stylet cuff is formed with a proximal annular portion and a distal annular portion, the proximal annular portion being sealably installed on the stylet wire assembly and the distal annular portion being sealably installed on the flexible guide tip, whereby fluid communication between the stylet cuff and the secondary lumen is achieved through the distal opening.

15. The apparatus of claim 11 further comprising a unidirectional end cap installed on the stylet wire assembly substantially at the proximal end.

16. The apparatus of claim 11 wherein:
the stylet wire assembly includes a stylet sheath having a primary lumen;
the inflation means comprises the primary lumen so as to communicate between the proximal end and the distal end of the stylet wire assembly.

17. An intubation stylet apparatus for use in medical intubation of a patient comprising:
a stylet wire assembly having a proximal end and a distal end;
a stylet cuff installed on the stylet wire assembly substantially at the distal end; and
a unidirectional end cap installed on the stylet wire assembly substantially at the proximal end.

18. The apparatus of claim 17 wherein the unidirectional end cap is formed with at least one living hinge oriented so as to allow distal movement of the unidirectional end cap along the stylet wire assembly and so as to discourage proximal movement of the unidirectional end cap along the stylet wire assembly.

19. The apparatus of claim 17 wherein:
a flexible guide tip is installed on the stylet wire assembly substantially at the distal end; and
the stylet cuff is formed with a proximal annular portion and a distal annular portion, the proximal annular portion being sealably installed on the stylet wire assembly and the distal annular portion being sealably installed on the flexible guide tip.

20. An intubation stylet apparatus for use in medical intubation of a patient comprising:
a stylet wire assembly having a proximal end and a distal end; and
a flexible guide tip installed on the stylet wire assembly substantially at the distal end, the flexible guide tip including a tip sheath having a hollow bore terminating in a distal closed end and further including a tip wire installed within the hollow bore.

21. The apparatus of claim 20 further comprising:
a stylet cuff installed on the stylet wire assembly and formed with a proximal annular portion and a distal annular portion, the proximal annular portion being sealably installed on the stylet wire assembly and the distal annular portion being sealably installed on the flexible guide tip; and
a means for inflating and deflating the stylet cuff.

22. The apparatus of claim 21 wherein:
the stylet wire assembly includes a stylet sheath;
the inflation means comprises a secondary lumen formed within the stylet sheath so as to communicate between the proximal end and the distal end of the stylet wire assembly; and the flexible guide tip is installed on the stylet wire assembly so that the distal opening of the secondary lumen is unobstructed, whereby fluid communication between the stylet cuff and the secondary lumen is achieved through the distal opening.

23. The apparatus of claim 20 further comprising a unidirectional end cap installed on the stylet wire assembly substantially at the proximal end.

24. The apparatus of claim 23 further comprising a stylet cuff installed on the stylet wire assembly substantially at the distal end.

25. A method of medically intubating a patient comprising the steps of:
inserting an intubation stylet within an intubation device;
inflating a stylet cuff installed substantially on the distal end of the intubation stylet so as to seat the intubation stylet within the intubation device and form a tapered transition from a distal flexible guide tip of the intubation stylet to the distal tip of the intubation device; and
passing the intubation stylet and intubation device into the trachea of the patient.

26. The method of claim 25, comprising the further step of engaging a unidirectional end cap mounted substantially on the proximal end of the intubation stylet with a connector installed substantially on the proximal end of the intubation device.

27. The method of claim 26 wherein the step of inserting the intubation stylet further comprises the steps of:
deflating the stylet cuff;
advancing the intubation stylet through the intubation device such that the flexible guide tip and the stylet cuff are temporarily passed out of the distal end of the intubation device until the unidirectional end cap engages the connector; and
retracting a stylet wire assembly of the intubation stylet relative to the unidirectional end cap until the stylet cuff is properly positioned adjacent the intubation device.

28. The method of claim 27 wherein the step of retracting the stylet wire assembly further comprises the step of aligning a mark on the intubation stylet with the distal tip of the intubation device.

29. The method of claim 26, comprising the further steps of:
inflating an intubation device cuff so as to seat and substantially seal the intubation device within the trachea;
deflating the stylet cuff; and
withdrawing the intubation stylet proximally by disengaging the unidirectional end cap from the connector.

30. The method of claim 25, comprising the further step of bending a stylet wire assembly of the intubation stylet into a desired configuration, whereby the intubation device conforms to the configuration of the intubation stylet for passing the intubation stylet and intubation device into the trachea of the patient.

31. The method of claim 25, wherein the step of passing the intubation stylet and intubation device into the trachea of the patient further comprises the steps of:
parting the larynx of the patient with the flexible guide tip of the intubation stylet; and
advancing the intubation device through the larynx and into the trachea as guided by the flexible guide tip of the intubation stylet.

32. A method of medically intubating a patient comprising the steps of:
inserting an intubation stylet within an intubation device;
advancing the intubation stylet through the intubation device such that a stylet cuff installed substantially on the distal end of the intubation stylet is temporarily passed out of the distal end of the intubation device;
engaging a unidirectional end cap mounted substantially on the proximal end of the intubation stylet with a connector installed substantially on the proximal end of the intubation device;
retracting a stylet wire assembly of the intubation stylet relative to the unidirectional end cap until the stylet cuff is properly positioned adjacent the intubation device;
inflating the stylet cuff so as to seat the intubation stylet within the intubation device and form a tapered transition from the distal tip of the intubation stylet to the distal tip of the intubation device;
bending the stylet wire assembly of the intubation stylet into a desired configuration, whereby the intubation device conforms to the configuration of the intubation stylet;
passing the intubation stylet and intubation device into the trachea of the patient;
inflating an intubation device cuff so as to seat and substantially seal the intubation device within the trachea;
deflating the stylet cuff; and
withdrawing the intubation stylet proximally by disengaging the unidirectional end cap from the connector.
